(19) United States
(12) Patent Application Publication
(10) Pub. No.: US 2022/0044827 A1
(43) Pub. Date: Feb. 10, 2022

(54) BIOLOGICAL INFECTION DETECTION METHOD, BIOLOGICAL INFECTION DETECTION AND PERSONNEL CONTROL METHOD AND BIOLOGICAL INFECTION RESPONSE SYSTEM

(71) Applicant: WEN-HAN CHANG, TAIPEI (TW)

(72) Inventor: WEN-HAN CHANG, TAIPEI (TW)

(21) Appl. No.: 17/350,056

(22) Filed: Jun. 17, 2021

(30) Foreign Application Priority Data
Aug. 7, 2020 (TW) 109126929
Jan. 20, 2021 (TW) 110102071

(51) Int. Cl. G16H 50/80; A61B 5/117; A61B 5/00; G16H 10/40; G16H 50/20

(52) U.S. Cl. CPC G16H 50/80; A61B 5/117; A61B 5/7264; G16H 50/20; A61B 5/48; G16H 10/40; A61B 5/746

(57) ABSTRACT

The present invention is provided a biological infection detection method, which includes the steps (a) is providing a screening kit with an identification code and a detection area; step (b) is binding an identity of a screened person and the identification code, wherein the identification code is binding the identity is defined as an associated identification code; step (c) is setting a biological sample of the screened person in the detection area; step (d) is generating a test result of the detected biological sample in the detection area; step (e) is to compare the associated identification code to ensure the test result belong to the screened person; and step (f), based on the test result, to determine whether the biological sample belongs to a first state, a second state, or an undeterminable state. The invention also provides a biological infection detection and personnel control method and biological infection response system.

### FIELD OF THE INVENTION

The present invention relates to a technical field of biological infection detection and control, especially a biological infection detection method, a biological infection detection and personnel control method, and a biological infection response system.

### BACKGROUND OF THE INVENTION

The environment is full of viruses that are harmful to the human body. When human beings have no resistance to the virus, it will endanger human life. In recent years, the pandemic caused by Severe Acute Respiratory Syndrome Coronavirus (SARS-CoV) and Severe Pneumonia with Novel Pathogens (COVID-19) has caused many human deaths.

Humans need to breathe in order to survive. Therefore, without any protective measures, viruses can easily enter the human respiratory tract through the air and infect humans; however, humans can reduce the risk of infection through methods such as vaccines and self-protection.

However, sometimes the virus has an incubation period so that the infected person does not know that he or she has been infected with the virus or the infected person is not affected by the virus but can spread the virus. If such an infected person cannot be detected or controlled in time, it will have a great impact on other healthy humans and cause a large number of infections.

In view of this, the present invention provides a biological infection detection method, a biological infection detection and personnel control method, and a biological infection response system, to solve the aforementioned problems.

### SUMMARY OF THE INVENTION

The first objective of the present invention is to provide a biological infection detection method, which is used to detect whether a screened person is infected, and to determine whether the biological sample belongs to the first state, the second state, or the undeterminable state based on the detection result.

The second objective of the present invention is to confirm the uniqueness of the screening result by binding the identification code and the identity of the screened person according to the aforementioned biological infection detection method.

The third objective of the present invention is to execute the artificial intelligence algorithm or image recognition algorithm to calculate the image of the detection result according to the aforementioned biological infection detection method, to determine that the biological sample of the screened person belongs to the first state, the second state or the undeterminable state.

The fourth objective of the present invention is based on the aforementioned biological infection detection method, by providing an electronic device to scan the identification code and the identity of the screened person (such as credentials, personal data, biological characteristics and/or behavioral characteristics, etc.), so that the screening kit is bound to the electronic device to form the associated identification code.

The fifth objective of the present invention is based on the aforementioned biological infection detection method to record the performer who collects the biological samples of the screened person and sets up the detection area (for example, the performer is the screened person himself or the non-screened person), for determining whether the biological sample is a trustworthy sample for testing.

The sixth objective of the present invention is based on the aforementioned biological infection detection method, wherein the electronic device provides an image capturing unit to capture the operation image of the performer, and in the process of capturing the operation image by the image capturing unit, loads the guiding image to guide the performer to obtain the biological sample, and compares the image difference between the guiding image and the operating image to determine whether the sampling process is accurate.

The seventh objective of the present invention is based on the aforementioned biological infection detection method, wherein if the performer is a non-screened person, the performer needs to provide a third-party certificate to set the biological sample of the screened person in the detection area, and if the performer fails to provide a third-party certificate, it will wait for the performer to provide the third-party certificate, let the screened person perform the test or cancel the test.

The eighth objective of the present invention is based on the aforementioned biological infection detection method, wherein when the detection result is determined to be the first state, the notification application is executed; when the detection result is determined to be the second state, the report application is executed; and, when the detection result is determined to be in an undeterminable state, the redetect application is executed.

The ninth objective of the present invention is based on the aforementioned biological infection detection method, requiring the screened person to provide auxiliary determination data, for example, travel history, medical history, contact history or health certificate to maintain non-abnormal information, or to re-execute the biological infection detection method during or after the incubation time.

The tenth objective of the present invention is based on the aforementioned biological infection detection method, wherein the report application can issue a pass certificate, and the pass certificate is optionally appended with a effective time to determine whether the certificate is invalid or valid; when the effective time expires, the pass certificate becomes invalidated.

The eleventh objective of the present invention is to perform a transfer procedure based on the aforementioned biological infection detection method, so that the screened person can be isolated and transported by a portable isolation device, so as to achieve the purpose of isolating the screened person from the external environment.

The twelfth objective of the present invention is to provide a screening kit (such as a biosensing chip or oily film) to capture the gas of the screened person based on the aforementioned biological infection detection method.

The thirteenth object of the present invention is to provide a biological infection detection and personnel control method used for detecting whether a screened person is infected and selectively implementing movement control for the screened person.

The fourteenth objective of the present invention is based on the aforementioned biological infection detection and personnel control method, selectively implementing movement control on the screened person according to the first state, the second state or the undeterminable state.

The fifteenth objective of the present invention is to delineate the activity range of the screened person on a virtual map based on the above biological infection detection and personnel control method and by comparing the screened person's coordinates in the actual geographic location to determine whether the screened person is in the activity range.

The sixteenth objective of the present invention is to mark the coordinates of the screened person in the first state or the undeterminable state on the virtual map based on the aforementioned biological infection detection and personnel control method, so as to selectively push and display such coordinates to any person or designated person.

The seventeenth objective of the present invention provides a biological infection response system which provides a screening kit with an identification code and a detection area to detect whether the screened person is infected and selectively implement movement control for the screened person.

In order to achieve the above objectives and other objectives, the present invention provides a biological infection detection method, which includes step (a) providing a screening kit with an identification code and a detection area; step (b) binding an identity of a screened person and the identification code so that the screening kit is associated with the identity, wherein the identification code bound to the identity is defined as an associated identification code; step (c) setting a biological sample of the screened person in the detection area, wherein the biological sample is obtained by directly sampling a gas, tissue or body fluid of the screened person, or the biological sample is obtained by further mixing with a reagent after the gas, tissue or body fluid of the screened person is collected; step (d) the detection area producing a test result of the tested biological sample; step (e) comparing the associated identification code to ensure the test result is of the person being screened; and step (f) determining according to the detection result that the biological sample belongs to a first state, a second state, or an undeterminable state.

In another embodiment, after the aforementioned step (f), further included are step (f1) when the detection result is determined to be the first state, executing a notification application to notify the preset designated object to carry out notifications and warnings, and selectively sending an abnormality message to the screened person; step (f2) when the detection result is determined to be the second state, executing a report application to send a non-abnormality message to the screened person; and step (f3) when the test result is determined to be undeterminable, a redetect application is executed to send a re-detect message for re-executing steps (a) to (f) to the screened person.

In another embodiment, the aforementioned report application additionally executes a transfer procedure. The screened person is isolated and transported by a portable isolation device. Wherein, the portable isolation device includes a protection unit for forming an accommodating space having an open end for accommodating the screened person. In addition, the protection unit further includes a protection piece and a support assembly. The supporting assembly is used for combining and propping up the protection piece. The support assembly includes two main support members and at least one movable support member. The two main support members are arranged opposite to each other, and at least one movable support member is connected to the two main support members. Wherein, at least one movable support member can move relative to the two main support members, so that the protection piece forms a detection area between the at least one movable support member and the two main support members, so that an external instrument can be used to detect the screened person through the protection piece.

In another embodiment, in the aforementioned step (c), when the biological sample is obtained in the form of gas, a carrier, a collection port and a carried object are further included. The carrier forms an accommodating space. The collection port is disposed at one end of the carrier. The collection port is for receiving gas. The carried object is disposed in the accommodating space to collect the target virus in the gas, and the carried object corresponds to the detection area.

In order to achieve the above and other objectives, the present invention provides a biological infection detection and personnel control method, which further includes step (a1) providing a screening kit with an identification code and a detection area; step (b1) binding the identification code to the identity of a screened person, so that the screening kit is associated with the identity, wherein the identification code of the bound identity is defined as an associated identification code; step (c1) setting the biological sample of the screened person in the detection area, wherein the biological sample is obtained by directly sampling a tissue or body fluid of the screened person, or the biological sample is obtained by further mixing with a reagent after sampling the tissue or body fluid of the screened person; step (d1) displaying a test result of the tested biological sample in the detection area; step (e1) comparing the associated identification code to confirm that it is the test result of the screened person; step (f1) determining based on the test result that the biological sample belongs to a first state, a second state, or an undeterminable state; and step (g1) selectively implementing an movement control on the screened person according to the first state, the second state, or the undeterminable state.

In order to achieve the above and other objectives, the present invention provides a biological infection response system, which provides a screening kit having an identification code and a detection area to detect whether a screened person is infected and selectively implement an movement control on the screened person, wherein the biological sample of the screened person is disposed in the detection area, and the biological sample is directly obtained from a tissue or body fluid of the screened person or the biological sample is obtained by further mixing with a reagent after sampling the tissue or body fluid of the screened person. The biological infection response system includes an input element and a processing element. The input element obtains the detection result displayed in the detection area, wherein the detection result is at least one of an image type and a text type. The processing element executes a biological infection detection application to bind the identification code and the identity of the screened person to associate the screening kit with the identity, and executes the biological infection detection application to determine whether the biological sample belongs to a first state, a second state, or a undeterminable state.

Compared with the conventional technology, the biological infection detection method, the biological infection detection and personnel control method, and the biological infection response system provided by the present invention can be performed by personnel qualified to collect biological samples on the screened person, or by the screened person to carry out the test on his or her own, and the results after the test can be used to determine whether the screened person is infected or not, and at the same time, it can be determined that the screening result belongs only to the person to be screened, which can be effectively determined that the screening result comes from the screened person. In addition, the present invention can also analyze the images of the screening results through the technology of image recognition to determine the screening results, such as positive, negative, or unclear. Furthermore, the present invention can issue pass certificates to screened persons with normal screening results or limit the range of activities of screened persons based on abnormal screening results, so as to effectively control the screened persons and avoid epidemics. diffusion. In addition, the present invention provides a biological infection response system to achieve the purpose of implementing biological infection detection and personnel control methods.

### BRIEF DESCRIPTION OF THE DRAWINGS

FIG. 1 is a flowchart of the biological infection detection method according to a first embodiment of the present invention.

FIG. 2 is a schematic diagram illustrating the structure of the screening kit of FIG. 1 of the present invention.

FIG. 3 is a another schematic diagram illustrating the structure of the screening kit of FIG. 1 of the present invention.

FIG. 4 is an implementation diagram illustrating the application of the screening kit of FIG. 3 of the present invention to a mask.

FIG. 5 is an implementation diagram illustrating the application of the screening kit of FIG. 3 of the present invention to an air conditioner.

FIG. 6 is a flowchart of the biological infection detection method according to a second embodiment of the present invention.

FIG. 7 is a schematic diagram illustrating the guiding image of the electronic device of FIG. 6 of the present invention.

FIG. 8 is a schematic diagram illustrating the structure of the portable isolation device for executing the report application in FIG. 1 of the present invention.

FIG. 9 is a three-dimensional schematic diagram illustrating the portable isolation device of FIG. 8 of the present invention.

FIG. 10 is a flowchart of the biological infection detection and personnel control method according to a third embodiment of the present invention.

FIG. 11 is a block diagram of a biological infection response system according to a fourth embodiment of the present invention.

FIG. 12 is a block diagram of the biological infection response system according to a fifth embodiment of the present invention.

### DETAILED DESCRIPTION OF THE PREFERRED EMBODIMENTS

Since various aspects and embodiments are only illustrative and non-limiting, after reading this specification, those with ordinary knowledge will know that other aspects and embodiments are possible without departing from the scope of the present invention. According to the following detailed description and the claims, the features and advantages of these embodiments will be more prominent.

In this specification, “a” or “an” is used to describe the elements and components described herein. This is just for the convenience of illustration and provides a general meaning to the scope of the present invention. Therefore, unless clearly stated otherwise, this description should be understood to include one or at least one, and the singular number also includes the plural number.

In addition, in this specification, the terms “include”, “have” or any other similar terms are intended to cover non-exclusive inclusions. For example, an element or structure containing a plurality of elements is not limited to the elements listed herein, but may include other elements that are not explicitly listed but are generally inherent to the element or structure.

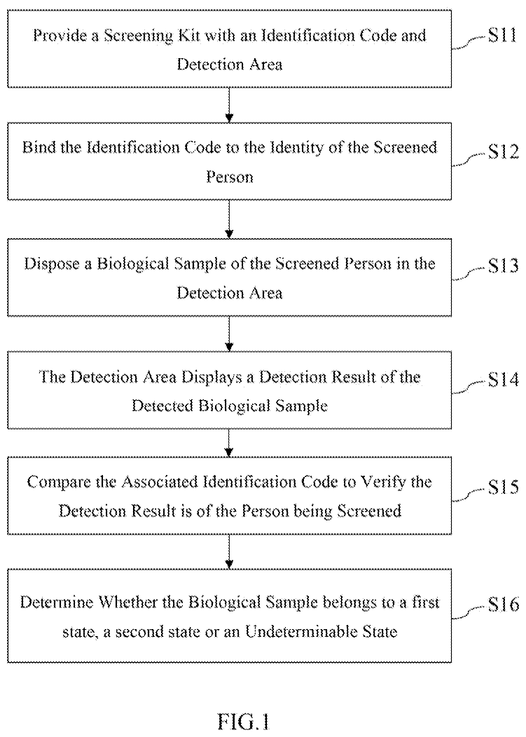

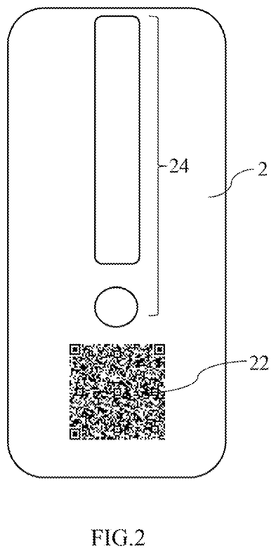

Please refer to FIG. 1, a flowchart of the biological infection detection method according to a first embodiment of the present invention. In FIG. 1, the biological infection detection method starts at step S11, which provides a screening kit with an identification code and a detection area. Refer also to FIG. 2, a schematic diagram illustrating of the structure screening kit in FIG. 1 of the present invention, for example, wherein the identification code can be a visible code such as a QR code, a barcode, a pattern code, or a non-visible RDIF code. In addition, the screening kit 2 in FIG. 2 provides an identification code 22 and a detection area 24, and the screening kit 2 in FIG. 2 is suitable for, for example, the tissue or body fluid of the screened person.

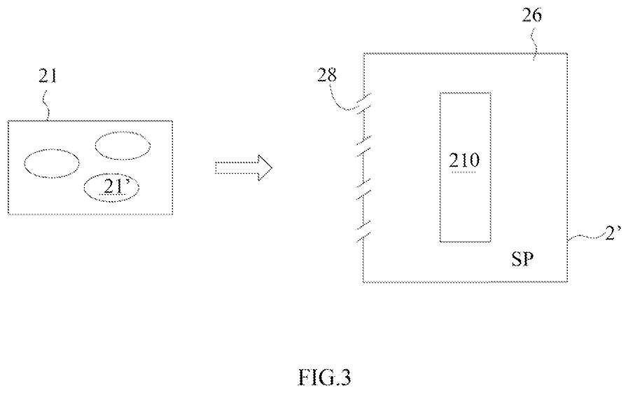

In another embodiment, refer also to FIG. 3, a schematic diagram illustrating another structure of the screening kit of FIG. 1 of the present invention. In FIG. 3, the screening kit 2′ can also be used to screen, for example, the person to be screened or the gas 21 in the environment. Wherein, the screening kit 2′ includes a carrier 26, including but not limited to masks, respirators, air conditioners, air ducts of air conditioners, breathing tubes or air supply tubes, air purifiers, etc. In addition, the carrier 26 is formed with an accommodating space SP, and one end of the carrier 26 is provided with at least one collection port 28 for receiving the gas 21. The screening kit 2′ further includes a carried object 210 disposed in the accommodating space SP, and the carried object 210 can be used to collect at least one target virus 21′ existing in the gas 21. One embodiment of the carried object 210 is a biosensing chip that has been modified to carry at least one aptamer molecule (Aptamer) that can bind to at least one target virus 21′, wherein aptamer molecule refers to oligonucleotide or peptide chain capable of binding a specific target molecule. In other words, the target virus 21′ is held on the biosensing chip by the aptamer molecule, and a corresponding electrical signal is generated by the number of the target virus 21′ collected, wherein the aptamer molecules mentioned are formed in at least one of carbon nanotube field-effect transistors, impedance components, or silicon nanowire field-effect transistors (SiNW-FET), but not limited thereto. The carbon nanotube field-effect transistors, for example, uses the special mechanical and charge conduction characteristics of carbon nanotubes to act in concert with the design of the circuit multiplexer in an array mode, successfully bonding to the comb-like structure of the aptamer molecule by modification, enabling it to have high sensitivity and specific selectivity. The silicon nanowire field effect transistor, for example, is a reusable SiNW-FET or a three-dimensional SiNW-FET. Moreover, the target virus 21′ includes but not limited to viruses such as novel coronavirus, type A or type B influenza virus, new H1N1 influenza virus, and H5N1 avian influenza virus. Herein, when the biosensing chip is in contact with the target virus 21′ in the gas 21, the aptamer molecule will combine with the target virus 21′ and generate a resistance current effect to generate electrical signals (including current and resistance). The voltage and current detection element (not shown in the figure) performs quantitative analysis on the electrical signal, thereby generating the corresponding voltage and current signal to understand the virus content of the target virus. In this way, one can instantly know whether he or she has been infected with target virus and the degree of infection. Furthermore, for example, using a warning component (not shown) to connect a voltage and current measuring component, wherein the warning component is used to output a warning notification drive signal for oneself to know or others to monitor the virus infection situation, the strength of the drive signal being related to the number or type of the target virus of 21′. Moreover, the implementation modes of the warning notification include but not limited to light warning, sound warning or notification warning. Take the light warning as an example, three levels of discrimination thresholds may be set, Level one being a red light warning when the virus content of a target virus exceeds 50%, level two being an orange light warning when the virus content of a target virus is 15-50%, and Level 3 being a green light warning when the virus content of a certain target virus is 0%, wherein the warning notice may also be a combination of multiple implementation modes. In another embodiment, there may be a plurality of biosensing chips, and different biosensing chips can be modified to have different aptamer molecules capable of sensing different target viruses 21′, thereby increasing the sensing efficiency of the screening kit 2′.

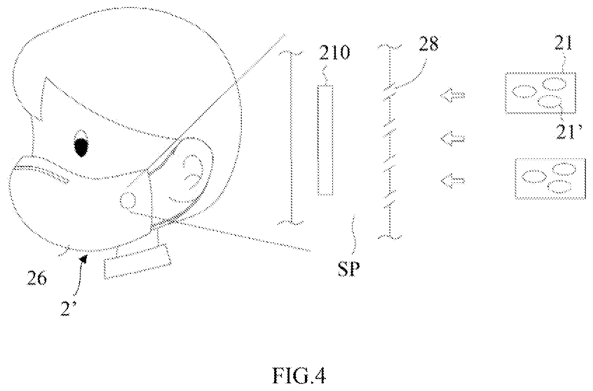

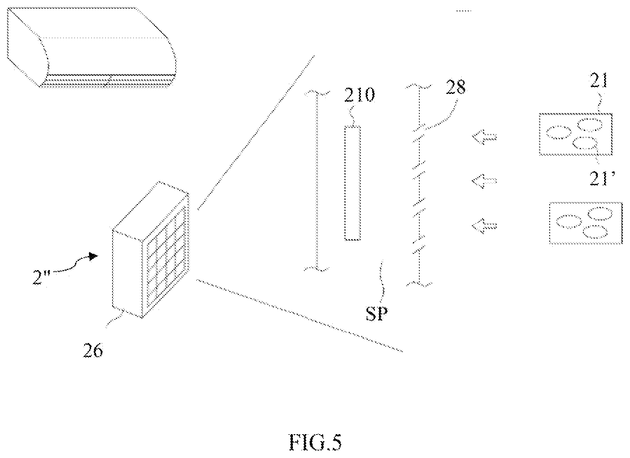

Referring also to FIG. 4, a schematic diagram illustrating the application of the screening kit of FIG. 3 of the present invention to a mask. In FIG. 4, the carrier 26 is a mask as an example, and the screening kit 2′ is a mask-type virus detection device with virus sensing function, wherein the accommodating space SP of the mask is provided with a carrier 210 that can capture the target virus 21′ in the gas 21 by means of an aptamer molecule; when the screened person wears a mask, if the target virus 21′ in the gas 21 enters through the collection port 28, it is quantified by the voltage and current detection element to generate a voltage and current signal, so it can be used to understand the virus content of the target virus 21′, and immediately know whether the target virus exists in the environment, achieving the effect of real-time monitoring of the virus and preventing spread of virus. Also, the carrier 26 may also be air-conditioning, which can be referred to in FIG. 5, a schematic diagram illustrating the application of the screening kit of FIG. 3 of the present invention to an air conditioner. In FIG. 5, a screening kit 2″ is embedded in the air-conditioner to detect viruses, where the virus detection process is as described above and will not be repeated.

In another embodiment, another aspect of the carried object 210 is an oily film (not shown). The oily film can be used to adhere the target virus 21′ to keep it on the oily film. When the embodiment of 210 is an oily film, the screening kit 2′ further contains a reagent (not shown) for acting on the oily film, so that the target virus 21′ is separated from the oily film and incorporated into the reagent. The reagent is then applied to the test paper having the aptamer molecule to display and determine the presence or absence of the target virus 21′.

Returning to FIG. 1, wherein step S12 is binding the identification code to the identity of the screened person, so that the screening kit is associated with the identity. For example, the screened person can refer to a natural person or a field, herein a natural person is taken as an example, wherein the identification code of the bound identity is defined as the associated identification code. It is worth noting that, in one embodiment, after the identification code is bound as the associated identification code, the identification code is prohibited from being bound again. Wherein, the binding method is as follows:

Method one: providing an electronic device to scan identification code, so that the screening kit is bound to the electronic device to form an associated identification code, wherein, the electronic device is pre-bound with the identity of the screened person. In other words, the electronic device may be owned by the screened person, and has been pre-bound to the screened person. For example, the electronic device may be a smart phone, tablet, etc., binding the identity of the screened person by using Subscriber Identity Module (SIM), Media Access Control Address (MAC address) or International Mobile Equipment Identity (IMEI) to enable a unique SIM, MAC address or IMEI to bind the screened person's identity.

Method two: providing an electronic device to scan the identification code and the identification certificate of the screened person, for example, the identification certificate can be a document such as an ID card, passport, etc. that can prove or record the screened person's identity, and by scanning the identification code and the identification certificate, the screening kit can bind the identification certificate to form an associated identification code, wherein the identification certificate is pre-bound with the identity of the screened person.

Method 3: providing an electronic device to scan the identification code and obtain the identity of at least one of personal data, biometrics and behavioral characteristics of the screened person, so that the screening kit is bound to the identity, biometrics, or behavioral characteristics to form an associated identification code. Wherein, personal data can be name, age, address, medical history, travel history, etc.; biological characteristics can be fingerprints, palm prints, palm shape, iris, facial features, voice prints, deoxyribonucleic acid (DNA); and, behavior features include walking posture, heartbeat, signature, gestures, etc.

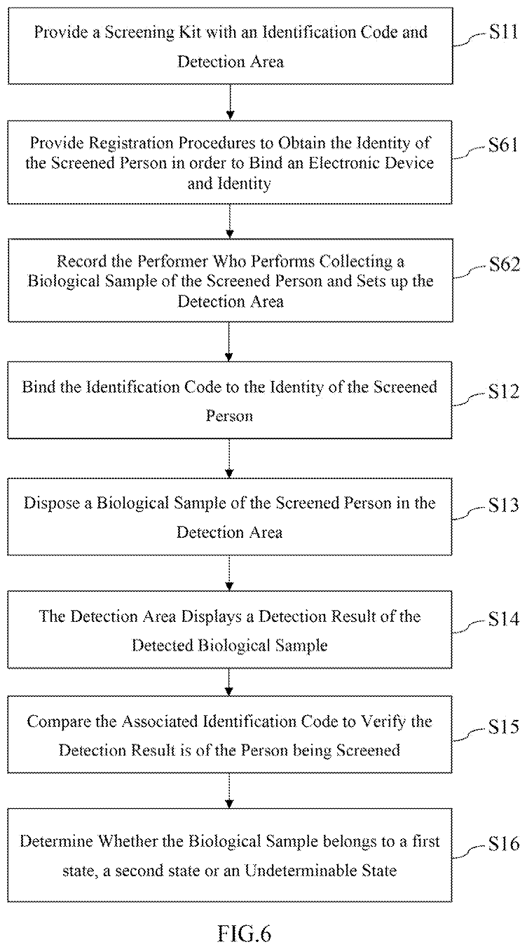

In the above method, step S61 may be performed before step S12. Referring also to FIG. 6, a flowchart of the biological infection detection method according to the second embodiment of the present invention. In step S61, a registration procedure is provided to obtain the identity of the screened person to bind the electronic device and the identity. For example, the registration procedure provides a webpage interface that can prompt the screened person to provide identity-related information and characteristics, such as name, age, address, medical history, travel history, fingerprints, palm prints, palm shape, iris, face, voice prints, DNA, walking posture, heartbeat, signature, gestures, etc., as well as one of the subscriber identity module, media access control address or international mobile device identification code of the electronic device used by the screened person or the biological characteristics of the screened person related to the identity.

Also, in another embodiment, the following steps may be performed before step S12 is performed to determine whether to perform step S12.

For example, providing a survey form for the screened person to fill out, wherein the survey form is at least one of personal data, travel history, contact history, and medical history, and the degree of completion of the survey form is assessed to determine the execution of step S12 and subsequent steps S13 to S16.

Returning to FIG. 1, in step S13, the biological sample of the screened person is disposed in the detection area. Wherein, the biological sample is obtained by directly collecting the gas, tissue or body fluid of the screened person, or the biological sample is obtained by further mixing with a reagent after collecting the gas, tissue or body fluid of the screened person, as described above and will be omitted herein.

Referring to FIG. 6, before step S13 is performed, step S62 can be performed, which is recording the performer who performed the sampling of the biological sample of the screened person and set up the detection area, for example, the performer is the screened person or a non-screened person, in terms of non-screened persons, may be doctors, nurses, and trained personnel, etc. The purpose of the record is to further determine whether the biological sample of the screened person is correct during the biological sample collection process, and only correct sampling can lead to correct detection result, and vice versa. In addition, besides in the form of text or pictures, the recording method can also be achieved by providing an image capturing unit to obtain images of the performer or images of the operation process, such as a camera or a camera lens of an electronic device.

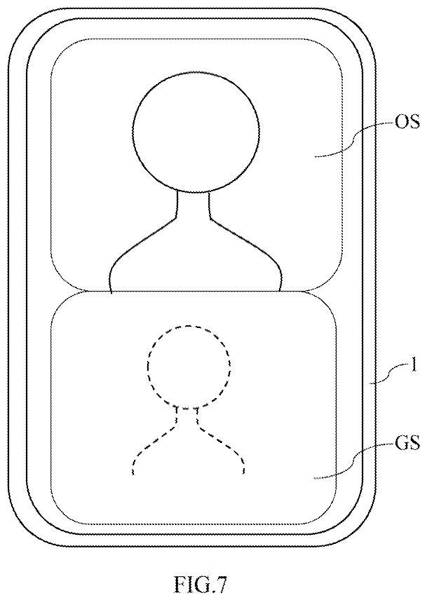

In another embodiment, in step S62, a text, picture or image for guiding the teaching may be provided to guide the performer, especially when the performer is the screened person. For example, the screened person can use the electronic device which provides an image capturing unit to capture the operation image of the operation process of the performer taking the biological sample, and at the same time display the guiding image on the electronic device to guide the performer to correctly take the biological sample. Therefore, guiding imagery is a method step related to the performer taking biological samples. Please also refer to FIG. 7, a schematic diagram illustrating the guiding image of the electronic device in FIG. 6 of the present invention. In FIG. 7, the electronic device 1, the guiding image GS, and the operation image OS are displayed. Moreover, in this embodiment, the image difference between the guiding image GS and the operation image OS is compared to determine whether it is necessary to perform steps S11 and S12 again.

It is worth noting that the aforementioned trained qualified personnel may further require the performer to provide a third-party certificate in step S13, and the third-party certificate needs be compared as a valid certificate before performing step S13; on the other hand, if the performer fails to provide a third-party certificate or the third-party certificate is invalid, it waits for the performer to provide a third-party certificate or fails to execute step S13. Regarding the third-party certificate, the third-party certificate for engaging in taking biological samples can be issued through the professional training and professional certification of a third-party organization. Third-party certificate can improve the accuracy of sampling and the correctness of interpretation.

Returning to FIG. 1, up next is step S14, which is generating a detection result of the detected biological sample in the detection area. In this step, there may further include a waiting time, which allows the biological sample to display the detection result in the detection area during the waiting time or within the waiting time by means of at least one of prompting and counting. In other words, the biological sample needs to have enough time to interact with the screening kit to show the correct test results. The waiting time is set according to the different screening kits. For example, the waiting time is tens of seconds, minutes, or tens of minutes. By means of prompts or counting, the performer can know how long it takes or calculate how long it takes to wait for the detection area to display the detection result.

Step S15 is comparing the associated identification codes to determine the test result belongs to the screened person. In this step, by determining the associated identification code, it can be determined that the detection result belongs to the screened person.

Step S16 is to determine whether the biological sample belongs to a first state, a second state, or an undeterminable state according to the detection result. This step can be further described as follows:

(1) when the detection result is determined to be the first state, the notification application is executed. The notification application is to notify and alert preset designated objects, as well as to selectively send out abnormal detection messages to the screened persons. Wherein, the first state may be positive, false positive and other states.

(2) when the detection result is determined to be the second state, the report application is executed. The report application is to send a non-abnormal detection message to the screened person. Wherein, the second state may be negative, false negative and other states.

(3) when the detection result is determined to be in an undeterminable state, the redetect application is executed. The redetect application is to send a re-detection message for re-executing steps S11 to S16 to the screened person. Wherein, the undeterminable state may be a state of being unable to correctly display a positive or negative state, a state of false positive and false negative, etc.

It is worth noting that the second state described herein may be negative or false negative because of no detection due to the incubation time (or the incubation period) or the sampling process is incorrect. In (2) above, the report application further requires the screened person to provide auxiliary determination data, such as travel history, medical history, contact history, activity tracking, health certificate, etc., to maintain non-abnormal message. In another embodiment, if the incubation time is already known, the screened person may be required to perform steps S11 to S16 again within or after the incubation time.

In addition, the report application can also issue a pass certificate, for example, a QR code, a barcode, a pattern code, etc. In another embodiment, the aforementioned pass certificate may be additionally attached with a effective time and when the effective time is exceeded, the pass certificate becomes invalid. In a practical way, the pass certificate can be managed through a server. The pass certificate may include an issuance time, cut-off time, number of extension, extension time and other settings, and the validity of the pass certificate can be determined by the aforementioned calculation of the time, such as the comparison between the current time and the cut-off time, and so forth.

In addition, the report application can also request the screened person to provide self-health management content from time to time or perform steps S11 to S16 to determine the extension of the effective time. For example, the self-health management content includes breathing, body temperature, smell, and taste, fatigue, asthma, etc.

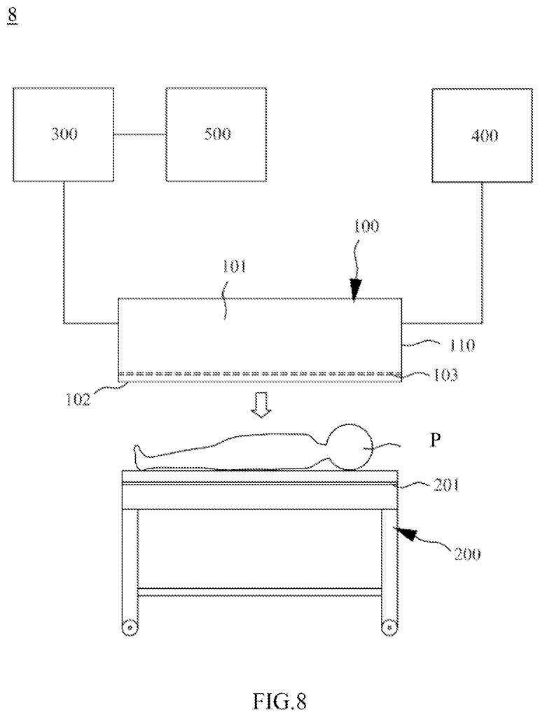

In another embodiment, the report application further includes a transfer procedure that can be executed to ensure that the screened person (or patient) can be effectively isolated from the external environment before arriving at the hospital. Referring also to FIG. 8, a schematic diagram illustrating the structure of a portable isolation device for executing the report application in FIG. 1 of the present invention. In FIG. 8, the screened person P is isolated and transported by the portable isolation device 8, where the portable isolation device 8 is mainly used to isolate the screened person P so that the screened person P can provide an effective isolation protection effect through the portable isolation device 8 during the transportation process. In FIG. 8, the portable isolation device 8 includes a protection unit 100 and a patient placement unit 200. The protection unit 100 is the main structure of the portable isolation device 8. The protection unit 100 is used forming an accommodating space 101 having an open end 102, so that the screened person P can enter the accommodating space 101 through the open end 102; that is, the protection unit 100 can accommodate the screened person P by the accommodating space 101. The patient placement unit 200 is used to carry the screened person P, and the screened person P can lie down or sit on the patient placement unit 200 for easy carrying by others. Here, the patient placement unit 200 can be an independent cushion structure (such as a mattress, etc.), or an assembly composed of the aforementioned cushion structure in combination with a trolley attached with a plurality of wheels, and the present invention is not limited thereto. Here, the patient placement unit 200 may include a corresponding sealing structure 201; the sealing structure 103 may be disposed near the open end 102 of the protection unit 100, and the corresponding sealing structure 201 may be disposed on each side of the patient placement unit 200. When the protection unit 100 can be disposed on the patient placement unit 200, the sealing structure 103 of the protection unit 100 is combined with the corresponding sealing structure 201 of the patient placement unit 200 to form an airtight structure, so that the aforementioned accommodation space 101 forms a closed space.

In addition, the portable isolation device 8 of the present invention further includes a gas processing unit 300. The gas processing unit 300 can be connected to the accommodating space 101 and the gas extraction unit 500 in the protection unit 100 via separate pipelines. That is to say, the gas in the accommodating space 101 will pass through the gas processing unit 300 first and then be discharged from the gas extraction unit 500 after being processed. Since the gas exhaled by the patient P may contain viruses, bacteria or microparticles, once the gas is discharged, there will be a risk of transmission. Therefore, the gas processing unit 300 is used to pre-process the gases to ensure the cleanliness and safety of the discharged gas. In an embodiment of the present invention, the gas processing unit 300 may be a nano-level air processor capable of decomposing viruses, bacteria, or microparticles by colliding with nano-level particles after being accelerated, but the present invention is not limited thereto, for example, the gas processing unit 300 may also be a filter with a function of adsorbing or filtering viruses, bacteria or microparticles.

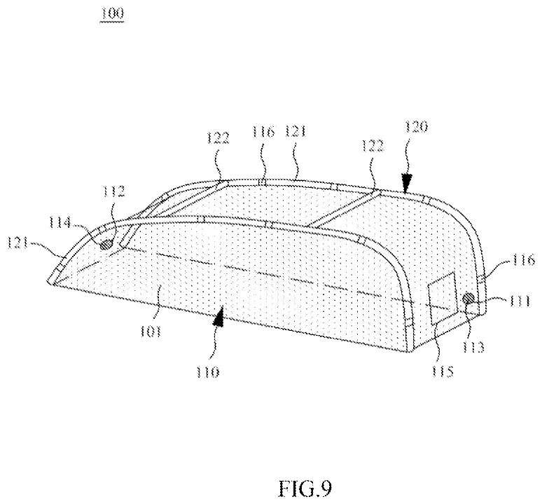

Referring also to FIG. 9, a three-dimensional schematic diagram illustrating the portable isolation device of FIG. 8 of the present invention. In FIG. 9, the protection unit 100 of the portable isolation device 8 includes a protection piece 110 and a support assembly 120. The protection unit 100 of the portable isolation device 8 uses the protection piece 110 as the main isolation structure, and the support assembly 120 is the main support skeleton structure of the protection unit 100. The support assembly 120 is used for combining and propping up the protection piece 110, so that the accommodating space 101 formed by the protection unit 100 maintains as much as possible a fixed size that can accommodate the screened person P. Here, when the protection unit 100 is supported and fully expanded, the height of the accommodating space 101 formed by the protection unit 100 is not higher than 60 cm, but the present invention is not limited thereto. Accordingly, the height of the accommodating space 101 of the aforementioned protection unit 100 can allow the screened person P to lie down or sit, and can be used with an ambulance or hospital emergency cart to facilitate the transportation of the screened person P. Wherein, the protection piece 110 is made of a transparent and flexible plastic material, such as plastic cloth, but other materials with similar characteristics can also be used instead. The transparent nature of the material makes it possible to directly observe the state of the patient in the protection unit 100 through the protection piece 110; the flexibility of the material facilitates the folding and storage of the protection piece 110 when not in use or after use, and the protection piece 110, when in use, can also provide the effect of relaxation and deformation in response to the detection of external equipment. In addition, the plastic material is lighter, making the protective piece 110 more convenient for handling. In another embodiment, the protection piece 110 includes an air inlet 111 for connecting with an air supply unit (not shown). The air inlet 111 may be arranged on the side of the protection piece 110, and the air inlet 111 and the air supply unit 400 are respectively connected to the two ends of an independent pipeline, so that the air supply unit 400 and the accommodating space 101 are gaseously connected with each other. Thereby, the gas supplied by the gas supply unit 400 will enter the accommodating space 101 through the air inlet 111. In one embodiment, the protection piece 110 further includes valve devices 113 and 114. The valve devices 113 and 114 are disposed at the location of the air inlet 111 where air enters. The valve devices 113 and 114 can be one-way valves or similar devices, and the valve devices 113 and 114 are in a closed state under normal conditions. Accordingly, when the air inlet 111 is not connected to the air supply unit, the valve devices 113 and 114 can be used to close the air inlet 111 to prevent the gas in the accommodating space 101 from leaking to the external environment; and when the pipeline of the air supply unit 400 is connected to the air inlet 111, the valve devices 113 and 114 can be opened with the insertion of the pipeline, causing the air supply unit 400 and the accommodating space 101 to be gaseously connected with each other.

The protective piece 110 further includes an air outlet 112 for connecting with the air exhaust unit 500. The air outlet 112 can be disposed on the side of the protection piece 110 (for example, on the opposite side where the air inlet 111 is provided) to facilitate gas flow, and the air outlet 112 and the air exhaust unit are respectively connected to the two ends of an independent pipeline (not shown in the figure), causing the air exhaust unit and the accommodating space 101 to be gaseously connected with each other. Thereby, the gas located in the accommodating space 101 will pass through the air outlet 112 and be drawn out by the air exhaust unit 500, enabling the accommodating space 101 to form a negative pressure isolation environment. In an embodiment of the present invention, the protection piece 110 further includes valve devices 113 and 114. The valve devices 113, 114 are disposed at the location of the air outlet 112 where the air exits. The valve devices 113 and 114 can be one-way valves or similar devices, and the valve devices 113 and 114 are in a closed state under normal conditions. Accordingly, when the air outlet 112 is not connected to the air exhaust unit 500, the valves 113 and 114 can be used to close the air outlet 112 to prevent the gas in the accommodating space 101 from leaking to the external environment; and when the pipeline of the air exhaust unit 500 is connected to the air outlet 112, the valve devices 113 and 114 can be opened with the insertion of the pipeline, causing the air extraction unit 500 and the accommodating space 101 to be gaseously connected with each other. Furthermore, in another embodiment, the protection piece 110 further includes at least one closable operating part 115. The operating part 115 can be disposed on any side of the protection piece 110, and the disposition thereof can correspond to the head or/and limbs of the patient when lying flat. When needed, medical staff can put their hands into the accommodating space 101 of the protection unit 100 via the operating part 115, so as to perform operations such as injection, drug administration, invasive intubation, or sputum extraction on the patient. The operating part 115 can adopt a cover with an automatic closing function or a component with an automatic tightening function, so that the operating part 115 is in a closed state under normal circumstances, and when the hand of the medical staff passes the operating part 115, the opening thereof can be reduced to the corresponding size of the hand to prevent the gas in the accommodating space 101 from leaking out as much as possible. If necessary, in an embodiment of the present invention, an independent isolation operation area may be additionally provided, and the isolation operation area may be provided between the accommodating space 101 and the operating part 115. The isolation operation area can provide the effect of double isolation. The medical staff can first extend their hands into the isolation operation area to perform medical pre-operations, and then further extend into the accommodating space 101 to perform corresponding medical operations on the patient.

In another embodiment, the protection piece 110 further includes a plurality of fixing structures 116 for assisting in combining and fixing corresponding parts of the support assembly 120. The plurality of fixing structures 116 can be bushings, collars or similar structural parts made of the same material as the protection piece 110, so that the plurality of fixing structures 116 and the protection piece 110 are integrally formed; the plurality of fixing structures 116 can also use stronger materials (such as metal or plastic material) made into the aforementioned structural parts, and are selectively disposed on the inner surface or the outer surface of the protection piece 110.

The support assembly 120 includes two main support members 121 and at least one movable support member 122. The two main support members 121 are disposed opposite to each other to respectively support the two sides of the protection piece 110, and a certain distance between the two main support members 121 for accommodating the patient is maintained. In an embodiment of the present invention, the aforementioned distance may correspond to the patient's height or body width, but the present invention is not limited thereto, and the distance can be adjusted according to different design requirements.

In order to enable the two main support members 121 to be quickly combined with the protection piece 110, in an embodiment of the present invention, the protective piece 110 further includes the aforementioned plurality of fixing structures 116, and the two main support members 121 can be combined with the protection piece 110 by the plurality of fixing structures 116. For example, if the fixing structure 116 is a collar, the user can pass each main support member 121 through the corresponding fixing structure 116 one by one until the main support member 121 passes through all the corresponding fixing structures 116 to be combined with the protection piece 110. At this time, the two main support members 121 can serve as a support frame of the protection piece 110, and the protection piece 110 can be propped up and unfolded.

Returning to FIG. 1, in step S16, it further includes performing an artificial intelligence algorithm or an image recognition algorithm to calculate the image of the detection result to determine whether the biological sample of the screened person belongs to the first state, the second state, or the undeterminable state.

In the foregoing, procedures such as notification applications, report applications, and redetect applications can be composed of program codes to drive the software or hardware of the electronic device to achieve the purpose of these applications.

In addition, in response to different fields of use, the aforementioned identity, identification code, associated identification code, detection result, first state, second state, undeterminable state, etc. can be stored in a local server and/or a cloud server. In addition, after the identification code is bound to form an associated identification code, by storing it in the local server and/or the cloud server, the identification code can be verified to prohibit binding thereof again on the local server and the cloud server.

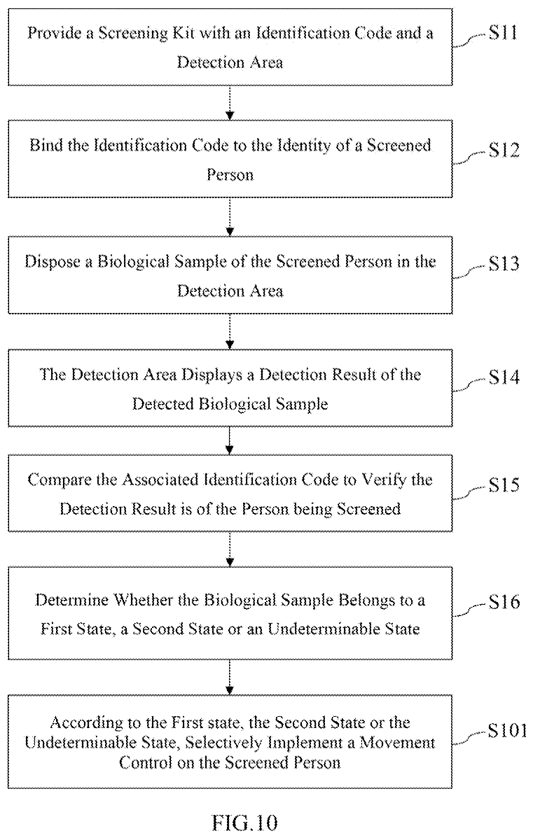

Please refer to FIG. 10, a flowchart of a biological infection detection and personnel control method according to a third embodiment of the present invention. In FIG. 10, the biological infection detection and personnel control method not only includes steps S11 to S16, but also includes step S101.

The description of steps S11 to S16 is the same as that described above and will not be repeated here.

Step S101 is to selectively implement an movement control on the screened person according to the first state, the second state or the undeterminable state. In this step, the screened person corresponding to the aforesaid states can be managed, controlled, monitored, etc. according to the first state, the second state, or the undeterminable state.

For example, the scope of activity of the screened person is delineated on the virtual map to establish a virtual electronic fence at the actual geographic location corresponding to the virtual map. After the virtual electronic fence is established, the coordinates of the screened person's actual geographic location are obtained, and the coordinates are compared with the virtual map to determine whether the screened person is in the virtual electronic fence, thereby specifically restricting or isolating those screened positive or undeterminable in the virtual electronic fence to avoid infecting other people.

In another embodiment, the screened person can also confirm the relationship between his or her own coordinates and the virtual electronic fence, that is, the screened person can determine his or her own scope of activities by displaying his or her own coordinates and the virtual electronic fence. In addition, by continuously recording the coordinates of the screened person, the movement track of the screened person can be established to provide an investigation of the path that the screened person has walked, so as to understand people, things, places and things the screened person may have been in contact with.

In another scenario, for the general public or those who are screened as negative, if they can know the location of the screened person who is positive or undeterminable, they can avoid going to or stay away from the location where the positive or undeterminable screened persons are present or the people, things, places and things they have been in contact with, and specific implementation methods can be exemplified as follows:

Method 1: if all screened persons who have completed the screening provide their own coordinates, so that these coordinates can be accompanied by the detection results, a safe distance between each other can be ensured. An example of a specific practice is to obtain the coordinates of each of the plural screened persons. Calculating the distance by performing algorithms on the first coordinates of the screened persons belonging to the first state or the undeterminable state and the second coordinates of the screened persons belonging to the second state; and determining that the distance is not greater than the predetermined safety distance, for example, the predetermined safety distance may be a few meters, and the warning application is further executed to notify the screened persons belonging to the first state or the undeterminable state and the screened persons belonging to the second state to keep at a predetermined safe distance or greater than the predetermined safe distance. Wherein, the execution of the warning application can send out an alarm to notify the screened persons in the second state that the distance between the screened persons in the first state or the undeterminable state is close or at a predetermined safe distance and should keep away; or, in another embodiment, it can be determined on the map which locations exist screened persons in the first state or the undeterminable state and therefore pose high risk, and the screened persons in the second state are reminded not go to locations marked with high risk on the map.

Method 2: marking the coordinates of the screened persons belonging to the first state or the undeterminable state on the virtual map to selectively push and display the aforesaid coordinates to any person or designated person.

In the process of implementing a movement control on the screened person, if the screened person needs help, the rescue application can be executed to send the location information of the screened person's electronic device to the preset recipients, for example, the recipients may be hospitals, fire stations, disease control departments, police stations, etc. For example, the rescue application can activate the dial-up function after the screened person executes the aforesaid rescue application, and automatically send location information for the recipient to find the screened person based on the location information. In another embodiment, the screened person can be kept where he or she is, and allow the hospital use the isolation cabin or fever tent be send the screened person to the hospital for examination.

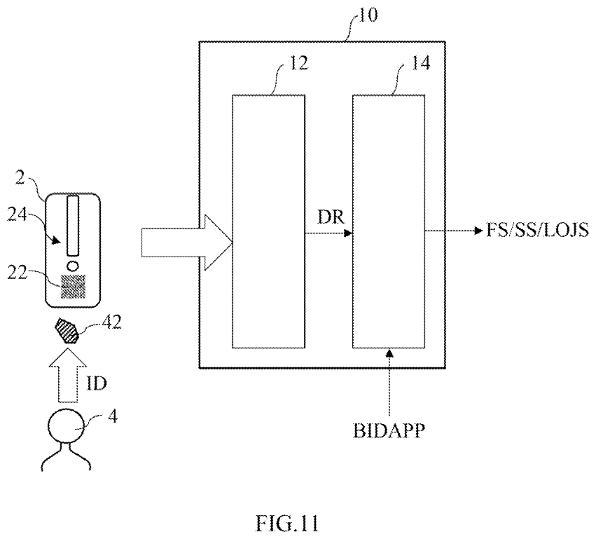

Referring to FIG. 11, it is a block diagram of a biological infection response system according to a fourth embodiment of the present invention. In FIG. 11, providing a biological infection response system 10 can provide a screening kit 2 having an identification code 22 and a detection area 24 to detect whether a screened person 4 is infected and selectively implement a movement control on the screened person 4. Wherein, the biological sample 42 of the screened person 4 is disposed in the detection area 24, and the biological sample 4 is obtained by directly collecting the gas, tissue or body fluid of the screened person 4 or the biological sample 42 is obtained by further mixing with a reagent after collection the gas, tissue or body fluid of the screened person. For example, the biological sample 42 can be gas, respiratory mucosa, saliva, cell membrane, exhaled air, droplets, tissue cells, DNA, etc. For example, taking the novel coronavirus (COVID-19) detection method as an example, the detection methods thereof include but not limited to nucleic acid detection, rapid serum antibody screening, and rapid antigen screening.

The biological infection response system 10 includes an input element 12 and a processing element 14.

The input element 12 obtains the detection result DR displayed in the detection area 24. Wherein, the detection result DR is at least one of an image type and a text type. For example, the input element 12 is an image capturing element, and the image capturing element scans the identification code 22 to provide to the processing element 14 to bind the identification code 22 and the identity ID of the screened person.

The processing element 14 executes a biological infection detection application BIDAPP to bind the identification code 22 and the identity ID of the screened person 4 to associate the screening kit 2 with the identity ID. The aforementioned embodiments can be referred to for the way that the biological infection detection application BIDAPP binds the identification code 22 and the identity ID.

Furthermore, the processing element 14 executing the biological infection detection application BIDAPP can further determine whether the biological sample 42 belongs to a first state FS, a second state SS, or an undeterminable state LOJS. Wherein for the first state FS, a second state SS, or an undeterminable state LOJS, reference can be made to the description in the foregoing embodiment.

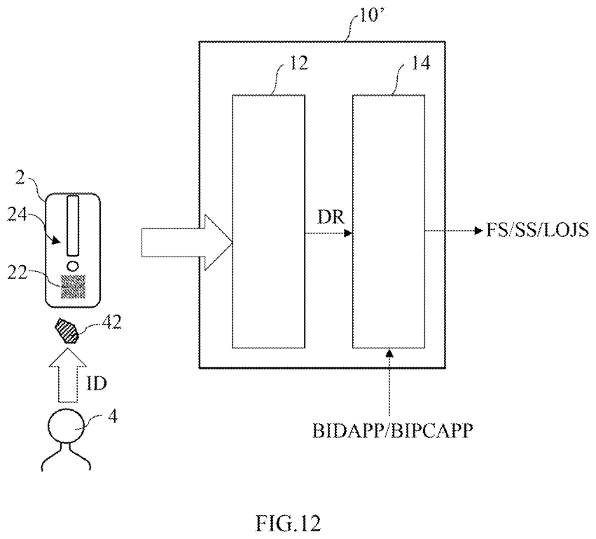

Referring to FIG. 12, a block diagram of a biological infection response system according to a fifth embodiment of the present invention. In FIG. 12, a biological infection response system 10′ is provided. In addition to the hardware components including the input element 12 and the processing element 14 of the fourth embodiment and the processing element 14 executing the biological infection detection application BIDAPP, the processing element 14 also executes biological infection personnel control application BIPCAPP to selectively implement movement control on the screened person 4 according to the first state FS, the second state SS, or the undeterminable state LOJS. For example, the biological infection control application BIPCAPP can delineate the scope of activities of the screened person on a virtual map (such as the map provided by Google, etc.), and establish a virtual electronic fence in the actual geographic location corresponding to the virtual map to control the screened person 4 in a virtual electronic fence. It is worth noting that the virtual electronic fence referred hereto does not actually generate a wall in a real geographic location, but is used to illustrate that after planning an activity scope on the virtual map, it can be understood that the activity scope surrounded by the boundary is composed of many point coordinates, and the continuous point coordinates form a line segment. When the biological infection personnel control application BIPCAPP calculates the coordinates of the screened person 4 and each coordinate or line segment of the sideline, the biological infection personnel control application BIPCAPP can determine whether the screened person 4's coordinates are adjacent to, on the boundary or crossing the boundary. Since the virtual map corresponds to the real geographic location, the determination on the virtual map can also be converted into the determination of the real geographic location. Therefore, to the screened person 4, an invisible virtual electronic fence exists in the real geographic location.

In another embodiment, the biological infection personnel control application BIPCAPP can, where the number of screened persons 4 are plural, further calculate first coordinates of the screened persons 4 belonging to the first state FS or undeterminable state LOJS and second coordinates of the screened persons 4 belonging to the second state SS, to calculate the distance between the first coordinates and the second coordinates, and when it is determined that the distance is not greater than the predetermined safe distance, the processing element 14 notifies the screened persons 4 belonging to the first state FS or the undeterminable state LOJS and the screened persons 4 belonging to the second state SS to maintain a predetermined safe distance or greater than a predetermined safe distance.

In another embodiment, the biological infection personnel control application BIPCAPP can calculate the first coordinates of the screened persons belonging to the first state FS or the undeterminable state LOJS and mark them on the virtual map, so as to selectively push and display to any person or designated person the first coordinates of the screened person with the first state FS or the undeterminable state LOJS. By pushing and displaying the first coordinates to any person or designated person, it is possible to prevent any person or designated person from moving to the screened person 4 with the first state FS or the undeterminable state LOJS, thereby reducing the risk of infection by the screened person 4 with the first state FS or the undeterminable state LOJS.

The above implementations are essentially only auxiliary descriptions, and are not intended to limit the embodiments of the subject matter or the applications or uses of the aforesaid embodiments. In addition, although at least one illustrative embodiment has been presented in the foregoing implementations, it should be understood that the present invention can still have a large number of changes. It should also be understood that the embodiments described herein are not intended to limit the scope, use, or configuration of the requested subject matter in any way. On the contrary, the foregoing embodiments will provide a convenient guide for those skilled in the art to implement one or more embodiments. Furthermore, various changes can be made to the function and arrangement of the components without departing from the scope defined by the scope of the patent application, and the scope of the patent application includes known equivalents and all foreseeable equivalents at the time of application of this patent application.

What is claimed is:

1. A biological infection detection method, including steps of:
(a) providing a screening kit having a identification code and a detection area;
(b) binding the identification code with the identity of a screened person, so that the screening kit is associated with the identity, wherein the identification code bound to the identity is defined as an associated identification code;
(c) disposing a biological sample of the screened person in the detection area, wherein the biological sample is obtained by directly sampling the gas, tissue or body fluid of the screened person, or the biological sample is obtained by further mixing with a reagent after sampling the gas, tissue or the body fluid of the screened person;
(d) the detection area producing the detection results of the detected biological sample;
(e) comparing the associated identification code to confirm that it is the detection result of the screened person; and
(f) determining according to the detection result that the biological sample belongs to the first state, the second state or the undeterminable state.

2. The biological infection detection method according to claim 1, wherein step (f) further includes:
(g) executing an artificial intelligence algorithm or an image recognition algorithm to compute an image of the detection result to determine that the biological sample of the screened person belongs to the first state, the second state or the undeterminable state.

3. The biological infection detection method according to claim 1, wherein step (b) further includes providing an electronic device to scan the identification code, so that the screening kit binds the electronic device to form the associated identification code, wherein the electronic device is pre-bound to the identity of the screened person.

4. The biological infection detection method according to claim 1, wherein step (b) further includes providing an electronic device to scan the identification code and at least one of the identity document, personal data, behavioral characteristics and biological characteristics of the screened person, so that the screening kit binds the identity document, personal data, behavioral characteristics, and biological characteristics to form the associated identification code.

5. The biological infection detection method according to claim 1, wherein prior to step (b), it further includes recording the performer who performed collecting of the biological sample of the screened person and setting up of the detection area, wherein the performer is the screened person or a non-screened person.

6. The biological infection detection method according to claim 5, further including a step of:
(h) providing an image capturing unit by means of an electronic device to capture the operation image of the operation process of the performer taking the biological sample, and simultaneously displaying a guide image on the electronic device to guide the performer to take the biological sample, wherein the guide image is related to the method steps for taking the biological sample by the performer.

7. The biological infection detection method according to claim 1, wherein step (c) further includes requesting the performer to provide a third-party certificate and determining to perform step (c) after comparing to verify the third-party certificate as a valid certificate.

8. The biological infection detection method according to claim 1, wherein after step (f), it further includes the steps of:
(i) when the detection result is determined to be the first state, executing the notification application to notify and alert the preset designated target and selectively send an abnormal detection message to the screened person;
(j) when the detection result is determined to be the second state, executing the report application to send a non-abnormal detection message to the screened person; and
(k) when the detection result is determined to be the undeterminable state, executing the redetect application to send a redetect message to the screened person to re-execute steps (a) to (f).

9. The biological infection detection method according to claim 8, wherein the report application issues a pass certificate, wherein the pass certificate is optionally appended with an effective time, and the pass certificate becomes invalid when the effective time expires.

10. The biological infection detection method according to claim 8, wherein the report application executes the transfer process, and the screened person is isolated and transported by a portable isolation device, wherein the portable isolation device includes:
a protection unit used for forming an accommodating space having an open end for accommodating the screened person, the protection unit further including;
a protection piece; and
a support assembly used for combining and propping up the protection piece, the supporting component including two main support members and at least one movable support member, the two main support members being arranged opposite to each other, and the at least one movable support member being connected to the two main support members;
wherein, the at least one movable support member can move relative to the two main support members, so that the protection piece forms a detection area between the at least one movable support member and the two main support members, thereby facilitating the use of an external instrument to detect the screened person through the protection piece.

11. The biological infection detection method according to claim 10, further including providing a gas processing unit respectively connected to an air outlet and an air exhaust unit of the protection piece to process the virus-carrying gas.

12. The biological infection detection method according to claim 1, wherein in step (c), when the biological sample is obtained from the gas, the screening kit further includes:
a carrier forming a accommodating space;
a collection port disposed at one end of the carrier, the collection port being used for receiving the gas; and
a carried object disposed in the accommodating space to collect the target virus of the gas, the carried object corresponding to the detection area.

13. The biological infection detection method according to claim 12, wherein the carried object is a biosensing chip or an oily film, providing aptamer molecules to bind the target virus to keep the target virus on the biosensing chip, and the oily film adheres the target virus to keep the target virus on the oily film.

14. The biological infection detection method according to claim 13, wherein step (d) further includes the biosensing chip generating the detection result of the corresponding electrical signal according to the number of the target virus collected by the biosensing chip.

15. The biological infection detection method according to claim 14, wherein the biosensing chip further includes an indicator element, and the indicator element generates a corresponding indicator signal according to at least one of the quantity and type of the target virus.

16. The biological infection detection method according to claim 13, further including a reagent acting on the oily film, causing the target virus to be separated from the oily film and incorporated into the reagent, wherein the reagent acts on a test paper having aptamer molecules to display the presence of the target virus.

17. A method for biological infection detection and personnel control, including steps of:
(a1) providing a screening kit having an identification code and a detection area;
(b1) binding the identification code with the identity of the screened person, so that the screening kit is associated with the identity, wherein the identification code bound to the identity is defined as an associated identification code;
(c1) disposing the biological sample of the screened person in the detection area, wherein the biological sample is obtained by directly sampling the tissue or body fluid of the screened person, or the biological sample is obtained by further mixing with a reagent after sampling the tissue or the body fluid of the screened person;
(d1) the detection area displaying the detection results of the biological sample tested;
(e1) comparing the associated identification code to confirm that the detection result is of the screened person;
(f1) determining according to the detection result whether the biological sample belongs to a first state, a second state, or an undeterminable state; and
(g1) selectively implementing movement control on the screened person according to the first state, the second state or the undeterminable state.

18. The method for biological infection detection and personnel control method according to claim 17, further including the steps of:
(h1) delineating the activity scope of the screened person on the virtual map to establish a virtual electronic fence at the actual geographic location corresponding to the virtual map;
(i1) obtaining the coordinates of the screened person in the actual geographic location; and
(j1) comparing the coordinates with the virtual map to determine whether the screened person is located in the virtual electronic fence.

19. A biological infection response system which provides a screening kit having an identification code and a detection area to detect whether a screened person is infected and selectively implement movement control on the screened person, wherein the biological sample of the screened person is disposed in the detection area, and the biological sample is obtained directly from the tissue or body fluid of the screened person or the biological sample is obtained by further mixing with a reagent after sampling the tissue or body fluid of the screened person, the biological infection response system including:
an input element for obtaining the detection result displayed in the detection area, wherein the detection result is at least one of an image type and a text type; and
a processing element for executing the biological infection detection application to bind the identification code and the identity of the screened person to associate the screening kit with the identity, and executing the biological infection detection application to determine that the biological sample belongs to a first state, a second state or an undeterminable state.

20. The biological infection response system according to claim 19, wherein the input element further includes scanning the identification code to provide to the processing element to bind the identification code to the identity of the screened person.

21. The biological infection response system according to claim 19, wherein the processing element further includes executing a biological infection personnel control application to selectively perform movement control on the screened person according to the first state, the second state or the undeterminable state

22. The biological infection response system according to claim 21, wherein the biological infection personnel control application further includes delineating the scope of activities of the screened person on the virtual map, and establishing a virtual electronic fence at the actual geographic location corresponding to the virtual map to control the screened person in the virtual electronic fence.

23. The biological infection response system according to claim 21, wherein the biological infection control application further includes computing, when the number of the screened persons is plural, a first coordinate of the screened persons belonging to the first state or the undeterminable state and a second coordinate of the screened persons belonging to the second state, so as to calculate the distance between the first coordinate and the second coordinate, and when the aforesaid distance is not greater than a predetermined safe distance, the processing element is used to notify the screened persons belonging to the first state or the undeterminable state the screened persons in the second state to keep at the predetermined safe distance or farther than the predetermined safe distance.
